(19) United States
(12) Patent Application Publication
(10) Pub. No.: US 2016/0361015 A1
(43) Pub. Date: Dec. 15, 2016

(54) MOISTURE WICKING ADHESIVES FOR SKIN-MOUNTED DEVICES

(71) Applicant: MC10, Inc., Lexington, MA (US)

(72) Inventors: Xianyan Wang, San Jose, CA (US); Roozbeh Ghaffari, Cambridge, MA (US); Pinghung Wei, Burlingame, CA (US); Ji Hyung Suzy Hong, Somerville, MA (US); Hakan Mutlu, North Chelmsford, MA (US); Brian Murphy, Medford, MA (US); David G. Garlock, Derry, NH (US)

(21) Appl. No.: 15/183,513

(22) Filed: Jun. 15, 2016

Related U.S. Application Data
(60) Provisional application No. 62/175,785, filed on Jun. 15, 2015.

(51) Int. Cl. A61B 5/00; B32B 3/16; B32B 27/10; B32B 27/18; B32B 27/08; B32B 7/12; B32B 27/26

(52) U.S. Cl. CPC A61B 5/6832; B32B 7/12; B32B 3/16; B32B 27/26; B32B 27/18; B32B 27/08; B32B 27/10; B32B 2307/726; B32B 2556/00; B32B 2535/00

(57) ABSTRACT

The present invention describes breathable multilayered adhesive structures that can be used as adhesives to mount devices onto the skin. The breathable multilayered adhesive structures can comprise moisture-wicking layers adhered to the skin by a porous adhesion layer. The pores allow moisture released from the skin to be transferred away through the moisture-wicking layer. The adhesive structures permit the devices to be skin-mounted for an extended period of time (e.g., a few hours or days) without causing moisture-associated skin injuries such as erythema, maceration, and irritation or inflammation.

### CROSS-REFERENCE TO RELATED APPLICATION

The present application claims the benefit of and priority to U.S. Provisional Application No. 62/175,785, entitled, “MOISTURE WICKING ADHESIVES FOR SKIN-MOUNTED DEVICES,” filed Jun. 15, 2015, the entirety of which is hereby incorporated by reference herein, including the drawings.

### STATEMENT REGARDING FEDERALLY SPONSORED RESEARCH

Not Applicable.

### REFERENCE TO MICROFICHE APPENDIX

Not Applicable.

### BACKGROUND

Technical Field of the Invention

This invention generally relates to adhesive structures for mounting devices onto the skin.

Description of the Prior Art

Some wearable devices are affixed to the skin using double-sided adhesive, which forms a non-breathable covering on the skin and prevents moisture (such as perspiration and vapor) from escaping. The adhesive can trap the moisture against the skin for a prolonged period of time, which can contribute to moisture-associated skin injuries, such as erythema, maceration, and irritation and inflammation. The moisture that accumulates also reduces the adhesion strength of the adhesive, causing detachment of the wearable devices from the skin, and affects the local sweating rate. The double-sided adhesive can also leave an unwanted residue on the skin and/or the wearable devices. In some cases, that site where the wearable device was affixed to skin of the user and/or the wearable device itself must be cleaned (e.g., wiped) to remove the residue or to reuse the wearable device.

### SUMMARY OF THE INVENTION

The invention relates to novel moisture-wicking adhesives and adhesive structures to improve the breathability and reduce the potential for injury. The moisture-wicking adhesives and adhesive structures can significantly reduce or prevent the buildup of moisture when a wearable device is mounted to the skin. In accordance with some embodiments, the adhesives can have a multilayered structure that can be configured to transfer moisture or sweat from the skin to the perimeter of the adhesive, preventing the accumulation that can contribute to injury.

In one aspect, the invention relates to a breathable multilayered adhesive structure comprising: (i) a moisture-wicking layer having a first surface and a second surface; (ii) a first adhesion layer having a first adhesion surface in contact with the first surface of the moisture-wicking layer and a second adhesion surface adapted to contact the skin, wherein the first adhesion layer includes a plurality of pores, enabling moisture from the skin to flow to the moisture-wicking layer.

In accordance with some embodiments of the invention, the moisture-wicking layer can be directly secured to the wearable device, such as by molding or bonding.

In accordance with some embodiments of the invention, the multilayered adhesive structure can include a second adhesion layer having a third adhesion surface in contact with the second surface of the moisture-wicking layer. And, the second adhesion layer can include a fourth adhesion surface in contact with wearable device.

In accordance with some embodiments of the invention, the wearable device can be selected from the group consisting of an electronic device, a photonic device, an optoelectronic device, or combinations thereof.

In accordance with some embodiments of the invention, the first surface and the second surface of the moisture-wicking layer can be substantially parallel.

In accordance with some embodiments of the invention, the first adhesion layer comprises a skin adhesive selected from the group consisting of silicone gel adhesive, a silicone pressure sensitive adhesive, an acrylic pressure sensitive adhesive, a natural or synthetic rubber adhesive, a hydrocolloid adhesive, and a hydrogel adhesive.

In accordance with some embodiments of the invention, the moisture-wicking layer comprises a plurality of microchannels and/or nanochannels adapted to promote moisture absorption and transfer (e.g., via capillary action).

In accordance with some embodiments of the invention, the moisture-wicking layer comprises a moisture-wicking material selected from the group consisting of absorbent paper, open-cell foam, non-woven fabrics, microfiber cloth, and nanofiber mesh.

In accordance with some embodiments of the invention, the first adhesion layer and the second adhesion layer each has a width, wherein the width of the first adhesion layer is greater than the width of the second adhesion layer.

In accordance with some embodiments of the invention, the moisture-wicking layer has a width greater than the width of the second adhesion layer.

In accordance with some embodiments of the invention, the first adhesion layer includes at least one cutout.

In accordance with some embodiments of the invention, the first adhesion layer comprises a plurality of disconnected segments.

In accordance with some embodiments of the invention, the breathable multilayered adhesive further comprises at least one through hole, thereby permitting the wearable device to be electrically connected to the skin.

In accordance with some embodiments of the invention, the moisture-wicking layer is 50 μm to 1.5 mm thick.

In accordance with some embodiments of the invention, the first adhesion layer is 15 μm to 500 μm thick.

In accordance with some embodiments of the invention, the second adhesion layer is 15 μm to 500 μm thick.

In accordance with some embodiments of the invention, at least a portion of the structure (e.g., the first adhesion layer) is flexible.

In accordance with some embodiments of the invention, at least a portion of the structure (e.g., the first adhesion layer) is stretchable.

In accordance with some embodiments of the invention, at least a portion of the structure (e.g., the first adhesion layer) is conformal.

In another aspect, the invention relates to an adhesive structure comprising: (i) a moisture-wicking layer having a first surface and a second surface, and (ii) a through hole extending through the first surface and the second surface, with a hydrogel portion within the through hole. The perimeter of the hydrogel portion penetrates into the moisture-wicking layer.

In accordance with some embodiments of the invention, the perimeter of the hydrogel portion penetrates about 1 to 1.5 mm into the moisture-wicking layer.

In accordance with some embodiments of the invention, the hydrogel portion has a first adhesion surface adapted to affix the adhesive structure to skin, and a second adhesion surface, opposite the first adhesion surface, adapted to affix the adhesive structure to a skin-mounted device.

In accordance with some embodiments of the invention, the structure further includes a first adhesion layer having a first adhesion surface in contact with the first surface of the moisture-wicking layer, and a second adhesion surface adapted to affix the adhesive structure to skin.

In accordance with some embodiments of the invention, the structure further includes a second adhesion layer having a third adhesion surface in contact with the second surface of the moisture-wicking layer, and a fourth adhesion surface adapted to affix the adhesive structure to an skin-mounted device.

In accordance with some embodiments of the invention, the fifth adhesion surface of the hydrogel portion and the second adhesion surface of the first adhesion layer are substantially co-planar, and the sixth adhesion surface of the hydrogel portion and the fourth adhesion surface of the second adhesion layer are substantially co-planar.

In accordance with some embodiments of the invention, the hydrogel portion is formed of a hydrogel precursor.

In accordance with some embodiments of the invention, the hydrogel precursor includes one or more monomers, one or more polymers, one or more crosslinking agents, one or more humectants, one or more electrolytes, and water.

In accordance with some embodiments of the invention, the one or more monomers include acrylic acid, a salt of acrylic acid, methacrylic acid, an acrylamide, 2-acrylamido-2-methylpropanesulfonic acid, a salt of 2-acrylamido-2-methylpropanesulfonic acid, dimethyl acrylamide, diacetone acrylamide butyl acrylate, or a combination thereof.

In accordance with some embodiments of the invention, the hydrogel precursor includes 1-25 wt % of the one or more monomers.

In accordance with some embodiments of the invention, the one or more polymers include polyvinylpyrrolidone, poly-2-acrylamido-2-methylpropanesulfonic acid, polyacrylic acid, polyvinyl alcohol, one or more ionic polyacrylamides, one or more non-ionic polyacrylamides, or a combination thereof.

In accordance with some embodiments of the invention, the hydrogel precursor includes 1-50 wt % of the one or more polymers.

In accordance with some embodiments of the invention, the one or more crosslinking agents include N,N′-methylene-bis-acrylamide, 1-hydroxycyclohexyl phenyl ketone, 2-hydroxy-2-methyl-1-phenyl-1-propanone, or a combination thereof.

In accordance with some embodiments of the invention, the hydrogel precursor includes 0.01-5 wt % of the one or more crosslinking agents.

In accordance with some embodiments of the invention, the one or more humectants include glycerol, propylene glycol, triethylene glycol, tripropylene glycol, butylene glycol, or a combination thereof.

In accordance with some embodiments of the invention, the hydrogel precursor includes 1-90 wt % of the one or more humectants.

In accordance with some embodiments of the invention, the one or more electrolytes include sodium chloride, potassium chloride, lithium chloride, or a combination thereof.

In accordance with some embodiments of the invention, the hydrogel precursor includes 0.1-25 wt % of the one or more electrolytes.

In accordance with some embodiments of the invention, the hydrogel precursor includes 1-95 wt % of the water.

In accordance with some embodiments of the invention, the hydrogel precursor includes one or more thickening agents.

In accordance with some embodiments of the invention, the one or more thickening agents include locust bean gum, cellulose, gelatin, agar, alginic acid, casein, collagen, guar gum, or a combination thereof.

In accordance with some embodiments of the invention, the hydrogel precursor includes of 20 wt % or less of the one or more thickening agents.

In accordance with some embodiments of the invention, the moisture-wicking layer includes a plurality of the through hole, and each through hole of the plurality of through holes includes within a hydrogel portion.

In another aspect, the invention relates to a method of forming an adhesive structure comprising: filling one or more through holes formed in a moisture-wicking layer with a hydrogel precursor, and curing the hydrogel precursor to form one or more hydrogel portions within the moisture-wicking layer. One or more properties of the hydrogel precursor, one or more characteristics of the filling, or a combination thereof cause the hydrogel precursor to penetrate into the moisture-wicking layer prior to the curing of the hydrogel precursor.

In accordance with some embodiments of the invention, the perimeters of the one or more hydrogel portions penetrate about 1 to 1.5 mm into the moisture-wicking layer.

In accordance with some embodiments of the invention, the hydrogel precursor has a viscosity of 2000-30000 cPs during the filling of the one or more through holes.

In accordance with some embodiments of the invention, the one or more characteristics of the filling include dispensing the hydrogel precursor at a dispenser head speed, a dispenser head height, a flow rate, or a combination thereof to cause the hydrogel precursor to penetrate the moisture-wicking layer.

In accordance with some embodiments of the invention, the curing of the hydrogel precursor comprises photopolymerization of the hydrogel precursor.

In accordance with some embodiments of the invention, the photopolymerization is electron beam photopolymerization, ultraviolet photopolymerization, or a combination thereof.

These and other capabilities of the invention, along with the invention itself, will be more fully understood after a review of the following figures, detailed description, and claims.

### BRIEF DESCRIPTION OF THE DRAWINGS

The accompanying drawing figures, which are incorporated into this specification, illustrate one or more exemplary embodiments of the inventions and, together with the detailed description, serve to explain and illustrate the principles and applications of these inventions. The drawings and detailed description are illustrative, and not limiting, and can be adapted and modified without departing from the scope and spirit of the inventions.

FIG. 1 is an illustration of a cross-section view of a moisture-wicking double-sided adhesive structure 100 in accordance with some embodiments of the invention.

FIG. 2 is an illustration of a cross-section view of a moisture-wicking double-sided adhesive structure 200 in accordance with some embodiments of the invention.

FIG. 3 is an illustration of a cross-section view of a moisture-wicking double-sided adhesive structure 300 in accordance with some embodiments of the invention.

FIG. 4 is an illustration of a cross-section view of a moisture-wicking double-sided adhesive structure 400 in accordance with some embodiments of the invention.

FIG. 5A is an illustration of a perspective view of a moisture-wicking double-sided adhesive structure 500 in accordance with some embodiments of the invention.

FIG. 5B is an illustration of a cross-section view of the moisture-wicking double-sided adhesive structure 500 of FIG. 5A in accordance with some embodiments of the invention.

FIG. 6A is an illustration of a perspective view of a moisture-wicking double-sided adhesive structure 600 in accordance with some embodiments of the invention.

FIG. 6B is an illustration of a cross-section view of the moisture-wicking double-sided adhesive structure 600 of FIG. 6A in accordance with some embodiments of the invention.

FIG. 7A is an illustration of a perspective view of a moisture-wicking double-sided adhesive structure 700 in accordance with some embodiments of the invention.

FIG. 7B is an illustration of a cross-section view of the moisture-wicking double-sided adhesive structure 700 of FIG. 7A along the line 7B-7B in accordance with some embodiments of the invention.

FIG. 7C is an illustration of a cross-section view of the moisture-wicking double-sided adhesive structure 700 of FIG. 7A along the line 7C-7C in accordance with some embodiments of the invention.

FIG. 7D is an illustration of a plan-view of a pattern formed by the movement of the dispenser tip during filling of the through hole 740 with a hydrogel precursor in accordance with some embodiments of the invention.

FIG. 7E is an illustration of a plan-view of another pattern formed by the movement of the dispenser tip during filling of the through hole 740 with a hydrogel precursor in accordance with some embodiments of the invention.

FIG. 7F is an illustration of a plan-view of another pattern formed by the movement of the dispenser tip during filling of the through hole 740 with a hydrogel precursor in accordance with some embodiments of the invention.

FIG. 7G is an illustration of a plan-view of another pattern formed by the movement of the dispenser tip during filling of the through hole 740 with a hydrogel precursor in accordance with some embodiments of the invention.

FIG. 7H is an illustration of a plan-view of another pattern formed by the movement of the dispenser tip during filling of the through hole 740 with a hydrogel precursor in accordance with some embodiments of the invention.

FIG. 7I is an illustration of a plan-view of another pattern formed by the movement of the dispenser tip during filling of the through hole 740 with a hydrogel precursor in accordance with some embodiments of the invention.

FIG. 7J is an illustration of a plan-view of another pattern formed by the movement of the dispenser tip during filling of the through hole 740 with a hydrogel precursor in accordance with some embodiments of the invention.

FIG. 8 is a flow chart of a process 800 for forming an adhesive structure with hydrogel portions in accordance with some embodiments of the invention.

FIG. 9A is an illustration of a top perspective view of a moisture wicking double-sided adhesive structure with electronic components in accordance with some embodiments of the invention.

FIG. 9B is an illustration of a bottom perspective view of the moisture wicking double-sided adhesive structure of FIG. 9A with electronic components in accordance with some embodiments of the invention.

FIG. 9C is an illustration of a bottom perspective view of an alternative moisture wicking double-sided adhesive structure with a wearable device in accordance with some embodiments of the invention.

### DETAILED DESCRIPTION OF THE ILLUSTRATED EMBODIMENTS

The present invention is directed to a porous adhesive structure having a moisture-wicking material that can absorb and transfer moisture away from the skin. The porous adhesive structure enables perspiration and vapor to be transferred through the moisture-wicking material and to escape from the perimeter of the adhesive (e.g., via capillary action). Accordingly, the present invention relates to adhesive structures, such as moisture-wicking double-sided adhesive structures. The moisture-wicking double-sided adhesive structure can be used to mount a wearable device, such as an electronic device, to a surface of a body, such as a human or animal body (e.g., on the skin), either directly (e.g., attached to the skin) or indirectly (e.g., attached to a covering layer, such as clothing, bandage, etc.). The moisture-wicking capability of the double-sided adhesive structure can prevent moisture or sweat from building up between the structure and the skin. Thus, the electronic device can be skin-mounted for an extended period of time (e.g., a few hours or days) without causing moisture-associated skin injuries such as erythema, maceration, and irritation or inflammation.

The moisture-wicking adhesive can be used to mount a wearable device for sensing, monitoring, and/or diagnosing. The device can be an electronic device, an optical device, an optoelectronic device, or any combinations thereof. As a non-limiting example, the example device can be a user-authentication, mobile-payment, and/or location-tracking electronic device. Other example applications include an accelerometer, a temperature sensor, a neuro-sensor, a hydration sensor, a heart sensor, a motion sensor, a flow sensor, a pressure sensor, an equipment monitor (e.g., smart equipment), a respiratory rhythm monitor, a skin conductance monitor, or any combinations thereof. In accordance with some embodiments of the invention, the wearable device can be in wireless communication with an external device such as a smart phone, a computer, a set-top box, an electronic pad or tablet, and a watch.

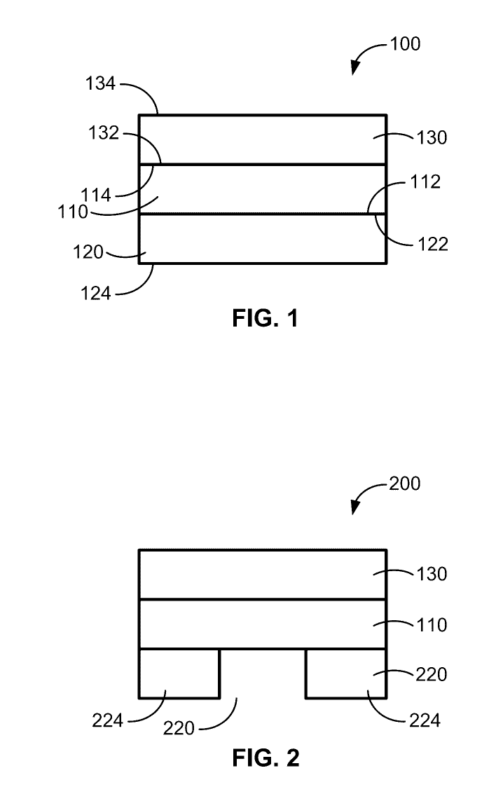

The moisture-wicking adhesive in accordance with some embodiments of the invention can be in the form of a multilayered structure. The moisture-wicking capability allows the adhesive to be breathable. FIG. 1 illustrates a cross-section view of a moisture-wicking double-sided adhesive structure 100 in accordance with some embodiments of the invention. The adhesive structure 100 can comprise a moisture-wicking layer 110, a first adhesion layer 120, and a second adhesion layer 130. The moisture-wicking layer 110 can have a first surface 112 and a second surface 114. The first surface 112 and the second surface 114 can be substantially parallel or non-parallel, such as in the case the moisture-wicking layer 110 having a varying thickness. The first adhesion layer 120 can have a first adhesion surface 122 in contact with the first surface 112 of the moisture-wicking layer 110 and a second adhesion surface 124 adapted to contact the skin. The second adhesion layer 130 can have a third adhesion surface 132 in contact with the second surface 114 of the moisture-wicking layer and a fourth adhesion surface 134 adapted to contact a wearable device, such as a skin-mounted device.

In accordance with some embodiments of the invention, the moisture-wicking layer 110 can comprise a moisture-wicking material. Examples of moisture-wicking materials include, but are not limited to moisture-wicking fabrics, absorbent papers, open-cell foams, and conductive nanofibrous films. The moisture-wicking fabrics can include microfiber and nanofiber based materials. The microfiber and nanofiber based materials can be made from polyesters, polyamides (e.g., nylon, KEVLAR®, NOMEX®, TROGAMID®), or a conjugation of polyester, polyamide, and polypropylene (PROLEN®). The moisture-wicking fabrics can be a knitted, woven, and non-woven fabric. The moisture-wicking fabrics can also be a blend of polyester, spandex, and nylon. Moisture-wicking fabrics can include materials available under the trade names: Dri-FIT, LUON®, LUXTREME®, Capilene, CoolMax, Coolpass, Fieldsensor, UnderArmour, MTS, and UA tech. Such moisture-wicking fabrics are typically used in athletic apparel.

In accordance with some embodiments of the invention, the moisture-wicking layer 110 can comprise a plurality of microchannels and/or nanochannels adapted to promote moisture absorption and transfer (e.g., via capillary action).

In accordance with some embodiments of the invention, the first adhesion layer 120 can include a plurality of pores, thereby enabling moisture from the skin to flow to the moisture-wicking layer 110. The plurality of pores can be distributed in a random or pre-determined pattern. In accordance with some embodiments of the invention, the first adhesion layer 120 can have a porosity of at least about 5%, at least about 10%, at least about 20%, at least about 30%, at least about 40%, at least about 50%, at least about 60%, at least about 70%, at least about 80%, at least about 90% or higher, but excluding 100%. In accordance with some embodiments of the invention, the porosity can range from about 5% to about 80%, or from about 10% to about 60%. The cross-section of the pores can have any width dimension provided that they permit moisture to pass through the first adhesion layer 120. In accordance with some embodiments, the first adhesion layer 120 can have pores arranged similarly to the way pores are arranged in an area of the skin or other tissue layer that the wearable device is to be applied. In accordance with some embodiments, the first adhesion layer 120 can have the same porosity as the skin or other tissue surface that the wearable device is to be applied. In accordance with some embodiments, the size of the pores in the first adhesion layer 120 can selected such that they include (e.g., overlie or cover) a predefined number of skin (or tissue) pores of the skin (or tissue) that the adhesive structure 100 is applied to.

The first adhesion layer 120 can comprise a skin adhesive which can be an unsupported transfer or double-coated adhesive. Any skin adhesives known in the art can be used in adhesives according to the invention. Suitable skin adhesives include acrylic-based, silicone-based, hydrocolloid-based, dextrin-based, and urethane-based adhesives, as well as natural and synthetic elastomers. Suitable examples include amorphous polyolefins (e.g., including amorphous polypropylene), KRATON® Brand synthetic elastomers, and natural rubber. Other exemplary skin adhesives include acrylic adhesives, cyanoacrylates, hydrocolloid adhesives, hydrogel adhesives, soft silicone adhesives, and silicone pressure sensitive adhesives. In accordance with some embodiments of the invention, the skin adhesive can comprise a silicone gel. In accordance with some embodiments of the invention, the skin adhesives can comprise an acrylic pressure sensitive adhesive. The first adhesion layer 120 can further comprise additives such as tackifiers, anti-oxidants, processing oils, and the like.

Less contact area between the first adhesion layer 120 and the skin can promote the comfort of wear and increase the rate of moisture transfer. For example, the first adhesion layer 120 can comprise at least one cutout (e.g., 1, 2, 3, 4, 5, 6, 7, 8, or more). The size of the cutout can be determined by ensuring that there is sufficient adhesion force between the first adhesion layer 120 and skin. The cutout can have any shape provided that there is sufficient adhesion force between the first adhesion layer 120 and the skin. For example, the cutout can be circular, oval, diamond, triangular, square, rectangular, pentagonal, or hexagonal. The second adhesion surface 124 of the first adhesion layer 120 can be textured to increase the adhesion force between the first adhesion layer 120 and the skin while reducing the contact area. For example, the second adhesion surface 124 can comprise a plurality of nanoscale or microscale pillars (e.g., see Mandavi et al., “A biodegradable and biocompatible gecko-inspired tissue adhesive,” Proc. Nat'l Acad. Sci., vol. 105, no. 7, Feb. 19, 2008, pp. 2307-2312, the disclosure of which is hereby incorporated by reference herein). The first adhesion layer 120 does not have to be a continuous layer or provide a continuous surface. In accordance with some embodiments of the invention, an adhesion layer can comprise a plurality of segments (e.g., 2, 3, 4, 5, 6, 7, 8, or more) that are either connected or disconnected.

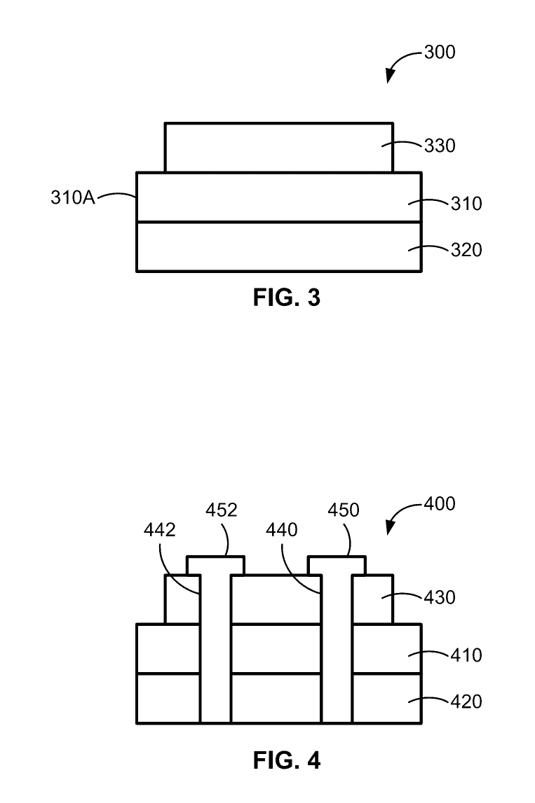

FIG. 2 is an illustration of a cross-section view of a moisture-wicking double-sided adhesive structure 200 in accordance with some embodiments of the invention. The moisture-wicking double-sided adhesive structure 200 is similar to that of the moisture-wicking double-sided adhesive structure 100 of FIG. 1. However, instead of the first adhesion layer 120, the moisture-wicking double-sided adhesive structure 200 includes the first adhesion layer 220. The first adhesion layer 220 includes a gap 222 between segments 224 of the first adhesion layer 220. Although only one gap 222 and two segments 224 are shown, there may be any number of gaps 222 and corresponding segments 224, while preserving the ability for the first adhesion layer 220 to adhere to a surface, such as skin.

Referring back to FIG. 1, in accordance with some embodiments of the invention, the moisture-wicking layer 110 can be attached to the wearable device by any attachment or bonding process. For example, the wearable device can have the moisture-wicking layer 110 molded into a surface or a portion thereof. In accordance with some embodiments, the moisture-wicking layer 110 can be bonded to the wearable device, such as by heat welding, ultrasonic bonding, solvent bonding, and/or plasma bonding. In accordance with some embodiments, the moisture-wicking layer 110 can be attached to the wearable device using an adhesive or glue. In accordance with some embodiments, a plastic layer comprising a plurality of holes can be disposed between the moisture-wicking layer 110 and the first adhesion layer 120. The wearable device together with the moisture-wicking layer 110 can be reusable.

The second adhesion layer 130 can include an adhesive which can be an unsupported transfer or double-coated adhesive. Any skin adhesives known in the art can be used in adhesives according to the invention. Suitable adhesives include acrylic-based, silicone-based, hydrocolloid-based, dextrin-based, and urethane-based adhesives, as well as natural and synthetic elastomers. Suitable examples include amorphous polyolefins (e.g., including amorphous polypropylene), KRATON® Brand synthetic elastomers, and natural rubber. Other exemplary skin adhesives include acrylic adhesives, cyanoacrylates, hydrocolloid adhesives, hydrogel adhesives, soft silicone adhesives, and silicone pressure sensitive adhesives. In accordance with some embodiments of the invention, the skin adhesive can comprise a silicone gel. In accordance with some embodiments of the invention, the skin adhesives can comprise an acrylic pressure sensitive adhesive. The second adhesion layer 130 can further comprise additives such as tackifiers, anti-oxidants, processing oils, and the like.

The thickness of the moisture-wicking layer 110 can be in the range of 50-1500 μm, such as 50-400 μm, or 100-350 μm. The thickness of the first adhesion layer 120 can be in the range of 15-500 μm. The thickness of the second adhesion layer 130 can be in the range of 15-500 μm.

In accordance with some embodiments of the invention, the second adhesion surface 124 can be in contact with a release liner. The fourth adhesion surface 134 can also be in contact with a release liner. The release liners enable the moisture-wicking adhesive structure 100 adhesive to be stored and transported without losing its adhesive properties or becoming contaminated.

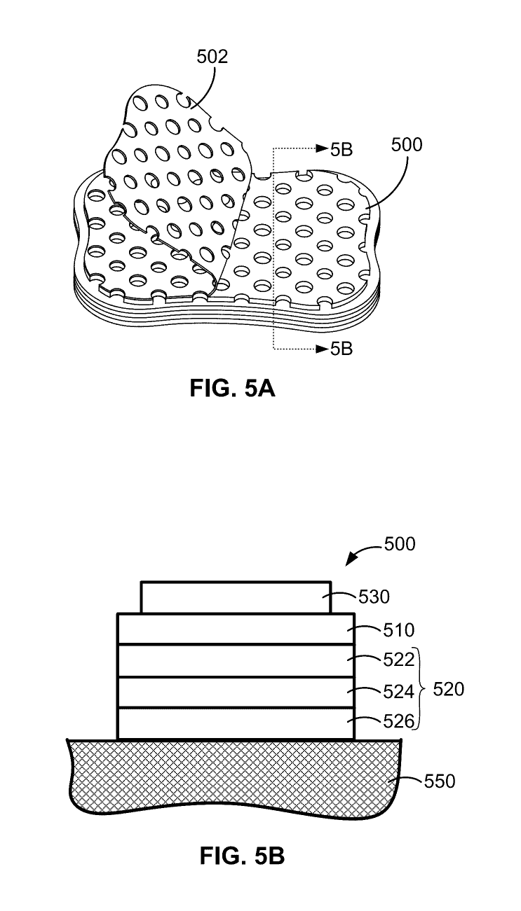

The widths of the moisture-wicking layer 110, the first adhesion layer 120, and the second adhesion layer 130 can be different from each other. FIG. 3 is an illustration of a cross-section view of a moisture-wicking double-sided adhesive structure 300 in accordance with some embodiments of the invention. The moisture-wicking double-sided adhesive structure 300 is similar to the moisture-wicking double-sided adhesive structure 100 of FIG. 1, except for the following differences.

In accordance with some embodiments of the invention, the width of the first adhesion layer 320 is greater than the width of the second adhesion layer 330, e.g., by 1-10 mm. In accordance with some embodiments of the invention, the width of the moisture-wicking layer 310 can be greater than the width of the second adhesion layer 330, e.g., by 1-10 mm. In accordance with some embodiments of the invention, the width of the moisture-wicking layer 310 can be greater than the width of the first adhesion layer 320, e.g., by 1-10 mm. Increasing the width of the moisture-wicking layer 310 exposes more of the moisture-wicking layer 310 (e.g., the outer edge 310A) to the ambient environment and enables moisture to evaporate faster out of the moisture-wicking layer 310, which facilitates (e.g., by capillary force) the transfer of moisture from the inner area to the edge of the moisture-wicking layer 310.

Referring back to FIG. 1, the moisture-wicking adhesive structure 100 can further comprise at least one through hole (e.g., 1, 2, 3 or more), thereby permitting components of the wearable device to directly contact the skin (e.g., electrodes, temperature sensors, acoustic actuators and transducers).

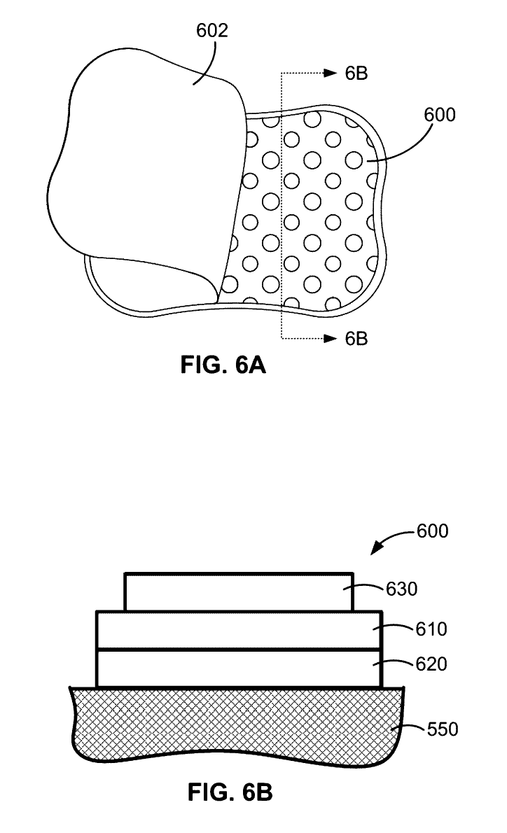

FIG. 4 is an illustration of a cross-section view of a moisture-wicking double-sided adhesive structure 400 in accordance with some embodiments of the invention. The double-sided adhesive structure 400 can comprise a moisture-wicking layer 410, a first adhesion layer 420, a second adhesion layer 430, a first through hole 440, a second through hole 442, a first electrode 450 extending into or through the first through hole 440, and a second electrode 452 extending into or through the second through hole 442. The first electrode 450 and the second electrode 452 can each be in physical and/or electrical contact with the wearable device in order to measure bio-potentials and bio-impedances (e.g., electrooculography (EOG), electroencephalography (EEG), electromyogram (EMG), galvanic skin response (GSR), and electrocardiogram (ECG) signals) and thermal signals (e.g., temperature and heat flux). The first through hole 440 and the second through hole 442 can each be filled with an electrically and/or thermally conductive material such as conductive hydrogels, metals, alloys, silver paste, carbon nanotube paste, and graphene paste. The ability of the wearable device to be electrically and/or thermally connected to the skin permits the wearable device to measure, sense, or monitor at least one parameter (e.g., heart rate, temperature, or hydration status).

The adhesives structures of the present invention can be fabricated using standard coating or lamination equipment known to those skilled in the art. The adhesive structures can be fabricated in the form of tapes, pads, patches, or other regular or irregular shaped adhesive elements, having any shape or size.

In accordance with some embodiments of the invention, at least a portion of the adhesive structure 100 can be flexible. For example, the first adhesion layer 120 can be flexible. In accordance with some embodiments of the invention, the adhesive structure 100 can take the form of a flexible double-sided adhesive structure or unsupported transfer adhesive.

In accordance with some embodiments of the invention, at least a portion of the adhesive structure 100 can be stretchable. For example, the first adhesion layer 120 can be stretchable. In accordance with some embodiments of the invention, the adhesive structure 100 can take the form of a stretchable double-sided adhesive structure or unsupported transfer adhesive.

In accordance with some embodiments of the invention, at least a portion of the adhesive structure 100 can be conformal. For example, the first adhesion layer can be conformal. In accordance with some embodiments of the invention, the adhesive structure 100 can take the form of a conformal double-sided adhesive structure or unsupported transfer adhesive.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

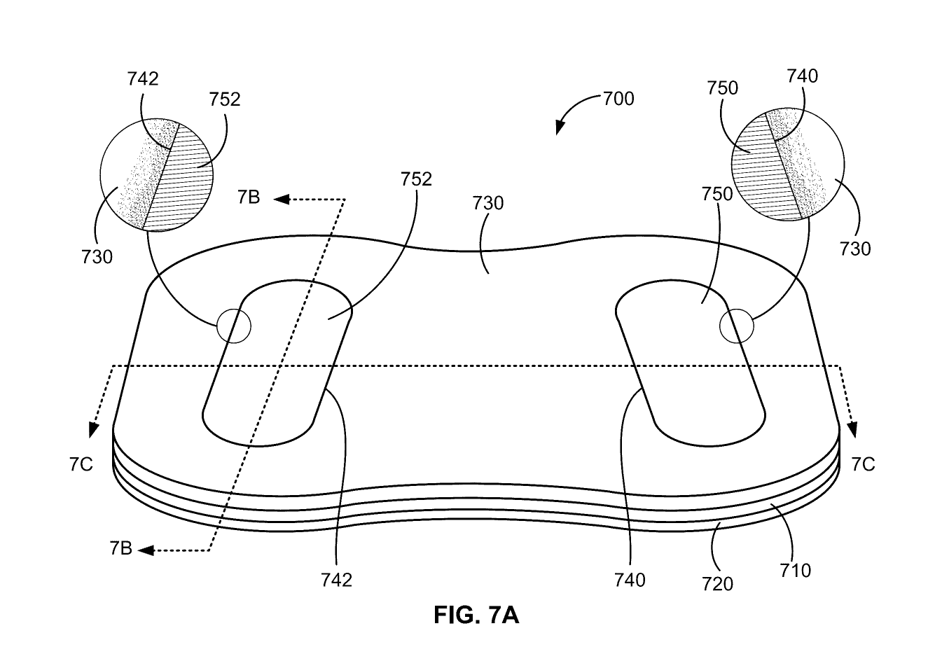

FIG. 5A is an illustration of a perspective view of an exemplary moisture-wicking double-sided adhesive structure 500, and FIG. 5B is an illustration of a cross-section view of the moisture-wicking double-sided adhesive structure 500 of FIG. 5A affixed to skin 550, in accordance with some embodiments of the invention. Generally, the moisture-wicking double-sided adhesive structure 500 includes a perforated first adhesion layer 520. The first adhesion layer 520 on the skin side can include a repositionable silicone gel adhesive and acrylic pressure sensitive adhesive. The moisture-wicking layer 510 can include a microfabric or micro-fiber based material. The second adhesion layer 530 can be positioned on the opposite side (e.g., the device side) of the moisture-wicking layer 510 from the first adhesion layer 520. The second adhesion layer 530 can include an acrylic transfer adhesive.

Referring to FIG. 5A, the moisture-wicking double-sided adhesive structure 500 can include removable release liners 502 covering the adhesion layers 520 and 530 prior to the moisture-wicking double-sided adhesive structure 500 being affixed to the skin of a user or a wearable device. FIG. 5A shows a removable release liner 502 partially removed, exposing the moisture-wicking double-sided adhesive structure 500 below.

Referring to FIG. 5B, in some aspects, the second adhesion layer 530 is the same shape and size as the wearable device to be adhered to the skin 550. However, the second adhesion layer 530 can be larger or smaller than the wearable device. The thickness of the second adhesion layer 530 can be about 2 mil.

The moisture-wicking double-sided adhesive structure 500 further includes the moisture-wicking layer 510 formed of the microfabric. The microfabric is 80% polyester and 20% nylon. The moisture-wicking layer 510 is about 2 mm larger than the perimeter of the second adhesive layer 530, as shown in FIG. 5B. The thickness of the moisture-wicking layer 510 can be about 0.35 mm.

The moisture-wicking double-sided adhesive structure 500 further includes the first adhesion layer 520. As shown, the first adhesion layer 520 can be formed of three separate layers, such that the first adhesion layer 520 can be a multilayered structure. The first adhesion layer 520 can include an acrylic adhesion layer 522 that can be about 1.6 mil thick. The first adhesion layer 520 can also include a polyurethane film layer 524 that can be about 1.5 mil thick. The first adhesion layer 520 can also include a silicone gel adhesion layer 526 that can be about 6 mil thick. The silicone gel adhesion layer 526 can be perforated with holes about 1.5-2.8 mm in diameter constituting about 17-29% openings. The first adhesion layer 520 can be about 2 mm larger than the perimeter of the second adhesive layer 530 and/or the wearable device, with full coverage or partial coverage options. The total thickness of the second adhesion layer 530 can be about 0.63 mm or thinner.

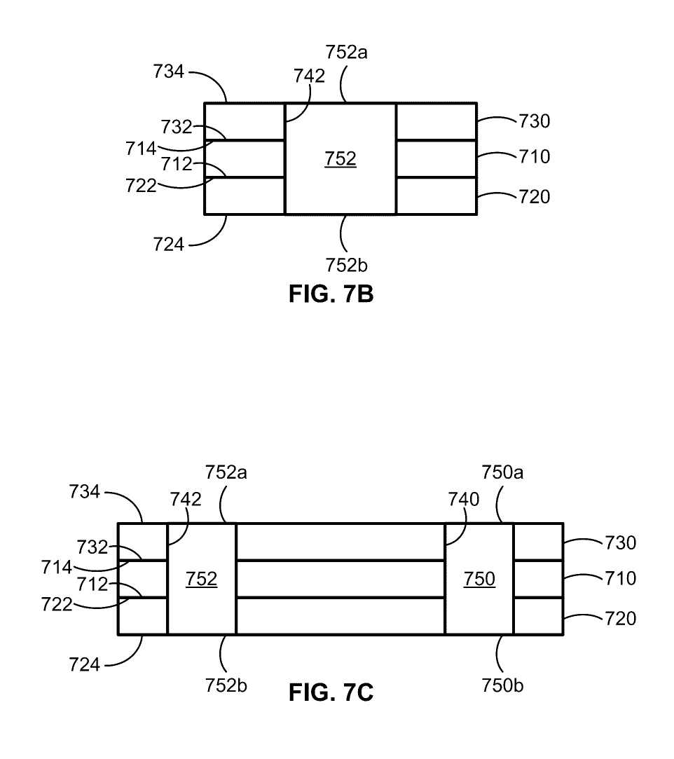

FIG. 6A is an illustration of a perspective view of another exemplary moisture-wicking double-sided adhesive structure 600, and FIG. 6B is an illustration of a cross-section view of the moisture-wicking double-sided adhesive structure 600 of FIG. 6A affixed to skin 550, in accordance with some embodiments of the invention. Generally, the moisture-wicking double-sided adhesive structure 600 includes a first adhesion layer 620 on the skin side of the adhesive structure 600 that includes a porous acrylic transfer pressure sensitive adhesive with 50% porosity (e.g., round, oval, rectangular, polygonal, and/or diamond shaped pores). The moisture-wicking layer 610 can include a microfabric or micro-fiber based material. The second adhesion layer 630 can be positioned on the opposite side (e.g., the device side) of the moisture-wicking layer 610 from the first adhesion layer 620. The second adhesion layer 630 can include an acrylic transfer adhesive.

Referring to FIG. 6A, the moisture-wicking double-sided adhesive structure 600 can include removable release liners 602 covering the adhesion layers 620 and 630 prior to the moisture-wicking double-sided adhesive structure 500 being affixed to the skin of a user or wearable device. FIG. 6A shows a removable release liner 602 partially removed, exposing the moisture-wicking double-sided adhesive structure 500 below.

Referring to FIG. 6B, the moisture-wicking double-sided adhesive structure 600 includes the second adhesive layer 630 formed of a transfer acrylic adhesive. In some aspects, the second adhesive layer 630 is the same shape and size as the wearable device to be adhered to the skin 550. However, the second adhesion layer 630 can be larger or smaller than the wearable device. The thickness of the second adhesion layer 630 can be about 2 mil.

The moisture-wicking double-sided adhesive structure 600 further includes the moisture-wicking layer 610 formed of the microfabric. The microfabric is 80% polyester and 20% nylon. The moisture-wicking layer 610 is about 2 mm larger than the perimeter of the second adhesive layer 630 and/or the wearable device. The thickness of the moisture-wicking layer 610 can be about 0.35 mm.

The moisture-wicking double-sided adhesive structure 600 further includes the first adhesion layer 620 formed of a patterned transfer acrylic adhesive that has a 50% diamond shape. The first adhesion layer 620 is 2 mm larger than the perimeter of the second adhesion layer 630 and/or the wearable device, with full coverage or partial coverage options. The thickness of the first adhesion layer 620 can be about 2 mil. The total thickness can be about 0.43 mm or thinner.

The moisture-wicking double-sided adhesive structures 500 and 600 were tested on 14 volunteers to assess skin redness, pain upon removal, and adhesion when attaching a wearable device to the chest for 2 to 3 days. Wear tests show significant reduction of skin redness and irritation and improved sweat tolerance when attaching wearable devices using the moisture-wicking adhesive materials of the prevent inventions in comparison with previously developed adhesives and other medical tapes. The skin area covered by the moisture-wicking double-sided adhesive structure 500 and 600 showed no noticeable redness, while a comparison adhesive of Tackwhite AR 11 induced noticeable red marks. The moisture-wicking double-sided adhesive structures 500 and 600 also withstood activities such as spinning, long distance running, ski, swimming, and other workouts that caused moderate to heavy sweating.

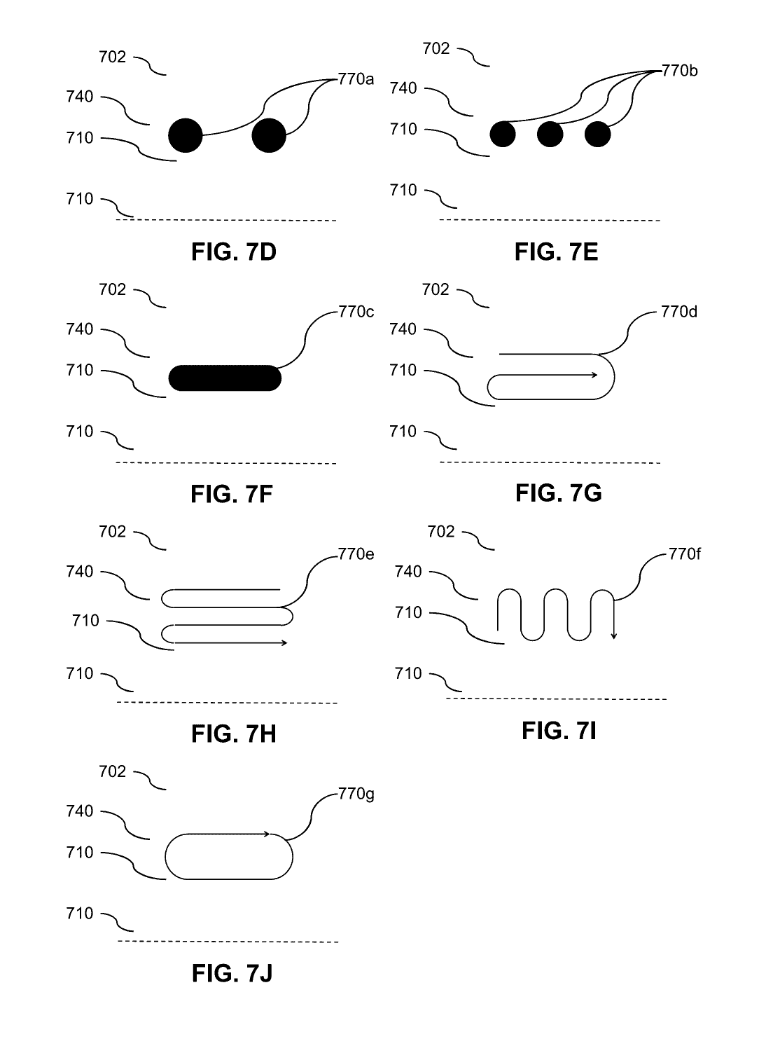

FIGS. 7A-7C show illustrations of a moisture-wicking double-sided adhesive structure 700 in accordance with some embodiments of the invention. Similar to the moisture-wicking double-sided adhesive structure 400 discussed above, the moisture-wicking double-sided adhesive structure 700 includes through holes to allow for a wearable device affixed to the moisture-wicking double-sided adhesive structure 700 to be in electrical contact with the skin upon which the moisture-wicking double-sided adhesive structure 700 is affixed.

Referring to FIG. 7A, the moisture-wicking double-sided adhesive structure 700 includes a moisture-wicking layer 710, a first adhesion layer 720, and a second adhesion layer 730, which are similar to the similarly numbered elements discussed above. These three layers (e.g., 710, 720, and 730) include two through holes 740 and 742 that extend therethrough, similar to the through holes 440 and 442. Although two through holes 740 and 742 are shown, the moisture-wicking double-sided adhesive structure 700 can include any number of through holes (e.g., 1, 2, 3, 4, 5, 6, 7, or more) depending on the size of the moisture-wicking double-sided adhesive structure 700 and the size of the through holes. The size and shape of the through holes 740 and 742 can also vary, such as the through holes 740 and 742 being oval, square, circular, rectangular, etc. in shape.

Within the through holes 740 and 742 are hydrogel portions 750 and 752. The hydrogel portions 750 and 752 entirely fill the through holes 740 and 742 and are filled with a hydrogel (e.g., a cured hydrogel precursor, as discussed below). As explained in greater detail below, the hydrogel portions 750 and 752 are mechanically, or mechanically and chemically, integrated into at least the moisture-wicking layer 710. Specifically, as shown in the callouts in FIG. 7A, the perimeters of the hydrogel portions 750 and 752 penetrate through the edges of the through holes 740 and 742. Depending on the porosity of the moisture-wicking layer 710, the hydrogel portions 750 and 752 can penetrate varying distances beyond the edges of the through holes 740 and 742. In some aspects, the hydrogel portions 750 and 752 can penetrate about 1-1.5 mm into the edges of the through holes 740 and 742. The penetration anchors the hydrogel portions 750 and 752 within the moisture-wicking double-sided adhesive structure 700. With the hydrogel portions 750 and 752 anchored or secured within the moisture-wicking double-sided adhesive structure 700, the moisture-wicking double-sided adhesive structure 700 can be affixed and removed from the skin of a user and a wearable device, and the hydrogel portions 750 and 752 can remain within the moisture-wicking double-sided adhesive structure 700. This quality can eliminate the need for a user to clean residual gel from the skin and the electrodes of the wearable device post-wear. Also, this quality is contrasted to electrodes (e.g., metal or metal alloy electrodes) placed within the through holes 740 and 742, which can come loose after use, particularly after repeated use, in addition to providing an inflexible and rigid structure that reduces the comfort of the overall feel of the moisture-wicking double-sided adhesive structure 700 on the user.

The hydrogel that is used to form the hydrogel portions has a cohesive strength and a rheological characteristic that enables the hydrogel to keep its shape without any supporting and/or reinforcing materials. Further, the hydrogel and hydrogel portions 750 and 752 are conductive, at least partially because of the water within the hydrogel. In some aspects, the hydrogel can include one or more other components that increase its electrical conductivity, such as salts, acids, etc. Thus, the hydrogel portions 750 and 752 provide electrically conductive interfaces between the skin (e.g., below the adhesion layer 720) and a wearable device (e.g., above the adhesion layer 730) and, therefore, allow for continuous signal recording and/or monitoring by the affixed wearable device.

In contrast with metals or metal alloys as the conductors within the through holes 740 and 742, the hydrogel portions 750 and 752 are soft, conformable, and skin-like conductors. The surfaces of the hydrogel portions 750 and 752 also provide for slightly wet and tacky electrically conductive interfaces. In contrast to metal electrodes, the hydrogel portions 750 and 752 also provide cushioning, while still also providing support and adhesion. Indeed, in some aspects, the hydrogel portions 750 and 752 can themselves provide adhesion to the skin and a wearable device. Depending on the surface area of the hydrogel portions 750 and 752 compared to the overall surface area of the moisture-wicking double-sided adhesive structure 700, one or both of the adhesion layers 720 and 730 can be replaced by solely the adhesion provided by the hydrogel portions 750 and 752. In these aspects, the moisture-wicking double-sided adhesive structure 700 can include only the hydrogel portions 750 and 752 anchored within the moisture-wicking layer 710. With respect to the skin side of the moisture-wicking double-sided adhesive structure 700, elimination of the adhesion layer 720 can increase the absorption of the moisture-wicking layer 710, as compared to the moisture-wicking double-sided adhesive structure 700 including the adhesion layer 720, while still providing the adhesion to the skin.

As compared to conventional electrodes, the hydrogel portions 750 and 752 can improve signal quality, user comfort and reduce the electrode dwell time. Dwell time is generally the period of time that a system or element of a system is required to remains in a given state to establish electrical communication between two elements. Non-hydrogel electrodes without any conductive medium between the electrode and the skin can require at least 10 to 15 minutes dwell time before the electrodes can be fully functional. In contrast, the electrical conductivity of the hydrogel eliminates the electrode dwell time upon contact with the skin. The elimination of the dwell time simplifies and shortens the preparation process required for users of the moisture-wicking double-sided adhesive structure 700 with an electrode device affixed to their skin.

Referring to FIGS. 7B and 7C, these figures show illustrations of cross-section views of the moisture-wicking double-sided adhesive structure 700 of FIG. 7A along the lines 7B-7B and 7C-7C, respectively, in accordance with some embodiments of the invention. Similarly numbered elements shown in FIGS. 7B and 7C correspond to similarly number elements described above. As shown, the hydrogel portions 750 and 752 include adhesion surfaces 750a and 752a that are co-planar with the adhesion surface 734 of the adhesion layer 730. Similarly, the hydrogel portions 750 and 7524 include adhesion surfaces 750b and 752b that are co-planar with the adhesion surface 724 of the adhesion layer 720. By being co-planar with the adhesion surface 724 of the adhesion layer 720, the hydrogel portions 750 and 752 can be in contact with the skin of the user upon which the moisture-wicking double-sided adhesive structure 700 is affixed for electrical contact with the skin. By being co-planar with the adhesion surface 734 of the adhesion layer 730, the hydrogel portions 750 and 752 can be in contact with the wearable device affixed to the moisture-wicking double-sided adhesive structure 700 for electrical contact with one or more electrodes of the wearable device.

FIGS. 7D-7J show plan-view illustrations of patterns formed by the movement of the dispenser tip during filling of the through hole 740 (and through hole 742, though not shown) with the hydrogel precursor in accordance with some embodiments of the invention. Each one of the patterns discussed below and illustrated can provide the above-described hydrogel precursor integration into the moisture-wicking layer 710. In all of FIGS. 7D-7J, and as discussed above, the through hole 740 extends through the moisture-wicking layer 710, the adhesion surface 730, and the adhesion surface 720 (not shown, behind the moisture-wicking layer 710).

Referring to FIG. 7D, the dispenser tip can dispense the hydrogel precursor at two locations within the through hole 740, as shown by the large dots 770a. For example, the dispenser tip can dispense approximately half of the hydrogel precursor when positioned according to one of the large dots 770a and dispense approximately the other half of the hydrogel precursor when positioned at the other one of the large dots 770a.

Referring to FIG. 7E, the dispenser tip can dispense the hydrogel precursor at three locations within the through hole 740, as shown by the small dots 770b. For example, the dispenser tip can dispense approximately one third of the hydrogel precursor positioned according to each one of the small dots 770b. The dispenser tip also can dispense the hydrogel precursor according to varying other numbers of dots, such as four, five, six, seven, eight, nine, or more dots, located throughout the through hole 740. The position of each of the dots can be random, evenly distributed throughout the through hole 740, or form a pattern, such as a five or six pointed star, one or more rings, etc.

Referring to FIG. 7F, the dispenser tip can dispense the hydrogel precursor while forming the pattern of a straight line along the length of the through hole 740, as shown by the thick line 770c.

Referring to FIG. 7G, the dispenser tip can dispense the hydrogel precursor while forming the pattern of an inward spiral, as shown by the line 770d. In some aspects, the dispenser tip alternatively can dispense the hydrogel precursor while forming the pattern of an outward spiral, which would be the reverse of the line 770d.

Referring to FIG. 7H, the dispenser tip can dispense the hydrogel precursor while forming a zig-zag pattern that reciprocates the dispenser tip along the length of the through hole 740, also referred to as a long zig-zag pattern, as shown by the zig-zag line 770e. Alternatively, and referring to FIG. 7I, the dispenser tip can dispense the hydrogel precursor while forming a zig-zag pattern that reciprocates the dispenser tip along the width of the through hole 740, also referred to as a short zig-zag pattern, as shown by the zig-zag line 770f.

Referring to FIG. 7J, the dispenser tip can dispense the hydrogel precursor while forming an oval pattern within the through hole 740, as shown by the oval 770g. The dispenser tip can move clockwise or counterclockwise.

The dispenser tip can dispense the hydrogel precursor according to other patterns not explicitly illustrated in the figures to integrate the hydrogel precursor into the moisture-wicking layer 710. In some aspects, the patterns shown in FIGS. 7D-7J can vary according to the shape of the through hole 740. For example, for a circular through hole, the dispenser tip can form a pattern of a circle, instead of an oval, in comparison to FIG. 7J. Similarly, for a square through hole, the dispenser tip can form a pattern of an inward or outward spiral that follows the perimeter of the square through hole 740, or that is circular, oval, or some other shape, in comparison to FIG. 7J.

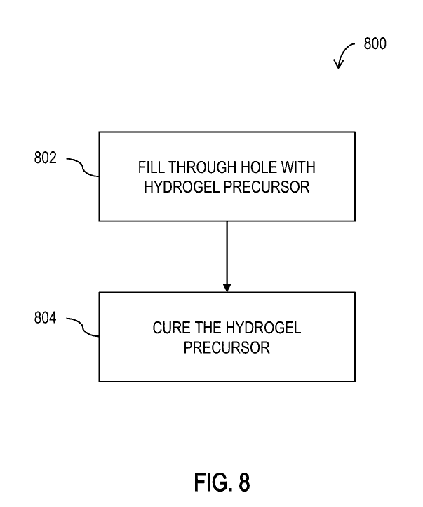

FIG. 8 is a flow chart of a process 800 for forming an adhesive structure, such as the moisture-wicking double-sided adhesive structure 700, with hydrogel portions 750 and 752 in accordance with some embodiments of the invention. The hydrogel portions 750 and 752 can be formed at various stages of forming the complete moisture-wicking double-sided adhesive structure 700. In some aspects, the hydrogel portions 750 and 752 can be formed after forming the multilayer structure of the adhesion layers 720 and 730 on opposite sides of the moisture-wicking layer 710. Moreover, the through holes 740 and 742 of the multilayered structure can be formed after forming the multilayered structure (e.g., after forming the adhesions layers 720 and 730 on the moisture-wicking layer 710). Alternatively, respective through holes can be formed in each of the moisture-wicking layer 710 and the adhesion layers 720 and 730 prior to forming the multilayered structure, such that the respective through holes line up to form the through holes 740 and 742 in the final multilayered structure.

Alternatively, the hydrogel portions 750 and 752 can be formed before forming the adhesion layers 720 and 730 on the moisture-wicking layer 710. Whether performed before or after, in both cases, at least the moisture-wicking layer 710 includes the through holes 740 or 742 (or respective through holes that eventually combine to form the through holes 740 and 742).

At step 802, the through holes 740 and 742 are filled with a hydrogel precursor. The hydrogel precursor is a non-crosslinked version of the final hydrogel before curing. The through holes 740 and 742 can be filled with the hydrogel precursor according to various methods, such as pouring, dispensing, and/or stenciling. The hydrogel precursor can have various chemistries while still providing a conductive contact for electrical communication through the moisture-wicking double-sided adhesive structure 700, in addition to providing an adhesive surface. The hydrogel precursor can include one or more monomers, one or more polymers, one or more crosslinking agents, one or more humectants, one or more electrolytes, and water.

The one or more monomers can include acrylic acid, a salt of acrylic acid, methacrylic acid, an acrylamide, 2-acrylamido-2-methylpropanesulfonic acid (AMPS), a salt of AMPS, dimethyl acrylamide, diacetone acrylamide butyl acrylate, carboxylic acids and their salts, sulfonic acids and their salts, or a combination thereof. The one or more monomers can constitute 1 to 25 wt % of the hydrogel precursor.

The one or more polymers can include polyvinylpyrrolidone (PVP), poly-2-acrylamido-2-methylpropanesulfonic acid, polyacrylic acid, polyvinyl alcohol (PVA), polyethylene oxide, hydroxyethyl methacrylate, polyacryl acetate, butyl acrylate, butyl methacrylate, ethyl acrylate, polyacrylates, one or more ionic polyacrylamides, one or more non-ionic polyacrylamides, or a combination thereof. The one or more polymers can constitute 1 to 50 wt % of the hydrogel precursor.

The one or more crosslinking agents can include N,N′-methylene-bis-acrylamide (nnMBA), 1-hydroxycyclohexyl phenyl ketone, 2-hydroxy-2-methyl-1-phenyl-1-propanone, 2,2-Dimethoxy-1,2-diphenylethan-1-one, 2-Benzyl-2-dimethylamino-1-(4-morpholinophenyl)-butanone-1, 1-[4-(2-Hydroxyethoxy)-phenyl]-2-hydroxy-2-methyl-1-propane-1-one, or a combination thereof. The one or more crosslinking agents can constitute 0.01 to 5 wt % of the one or more crosslinking agents.

The one or more humectants can include glycerol, propylene glycol, triethylene glycol, tripropylene glycol, butylene glycol, sorbitol, polyethylene glycol 400 (PEG 400), polyethylene glycol 600 (PEG 600), erythritol, phytantriol, varieties of diols and/or triols, or a combination thereof. The one or more humectants can constitute 1 to 90 wt % of the hydrogel precursor.

The one or more electrolytes can include sodium chloride, potassium chloride, lithium chloride, or a combination thereof. The one or more electrolytes can constitute 0.1 to 25 wt % of the hydrogel precursor. The remainder of the hydrogel precursor can include 1 to 95 wt % of water.

In some aspects, the hydrogel precursor can optionally include one or more thickening agents. The one or more thickening agents can include locust bean gum, cellulose, gelatin, agar, alginic acid, casein, collagen, guar gum, or a combination thereof. If present, the one or more thickening agents can constitute up to about 20 wt % of the hydrogel precursor.

At the time of filling the through holes 740 and 742 with the hydrogel precursor, the properties of the hydrogel precursor cause it to penetrate into the moisture-wicking layer 710. As described above, depending on, for example, the porosity of the moisture-wicking layer 710, the hydrogel precursor can penetrate about 1 to 1.5 mm into the moisture-wicking layer 710. In some aspects, a viscosity of hydrogel precursor of about 2000-30000 cPs during filling causes the hydrogel precursor to penetrate into the moisture-wicking layer 710 the 1 to 1.5 mm depth.

Additional characteristics of the filling of the hydrogel precursor, including dispensing location and pattern, dispenser head speed and height, and hydrogel precursor flow rate during dispensing can be tuned to have proper hydrogel precursor integration into the moisture-wicking layer 710. In some aspects, a flow rate of the hydrogel precursor between (and including) 1 to 100 milliliters per minute (ml/min), a dispenser tip height from the surface of the moisture-wicking layer 710 between (and including) 1 to 50 mm, and a dispenser tip speed between (and including) 1 to 100 centimeters per minute (cm/min) can integrate the hydrogel precursor into the moisture-wicking layer 710, such as to a depth of 1-1.5 mm into the moisture-wicking layer 710. As discussed above, the specific pattern that the dispenser tip forms during dispensing can also integrate the hydrogel precursor into the moisture-wicking layer 710. In some aspects, one side of the moisture-wicking double-sided adhesive structure 700 can include a removable release liner (e.g., removable release liner 502 or 602) that acts as a base or support for the hydrogel precursor during the filling and subsequent curing process. The removable release liner can prevent or limit the hydrogel precursor from flowing out of the through holes 740 and 742 before curing. Alternatively, another surface, besides a removable release liner, can be used as the base or support for the hydrogel precursor during the filling and curing process. Such other surfaces include, for example, a fixed or movable (e.g., conveyor belt) base or substrate upon which the moisture-wicking double-sided adhesive structure 700 is formed.

At step 804, the hydrogel precursor is cured to form the hydrogel and the hydrogel portions 750 and 752 portions integrated within the through holes 740 and 742. Curing the hydrogel precursor causes mechanical and/or chemical crosslinking of the components within the hydrogel precursor, such as crosslinking between monomers and oligomers, which creates longer polymer chains and affects the physical properties of the resulting hydrogel. Such physical properties include elasticity, viscosity, solubility, molecular weight, toxicity, etc. For example, the crosslinking increases the viscosity of the hydrogel precursor and forms the cured hydrogel.

The hydrogel precursor before and after curing (e.g., before and after physical and/or chemical crosslinking) exhibits two completely different flow characteristics. The hydrogel precursor behaves like a non-Newtonian fluid before curing. Thus, the hydrogel precursor is viscous under static conditions and less viscous when shear stress applied. As such, the viscosity of hydrogel precursor is dependent on shear rate, which allows the hydrogel precursor to flow into the through holes 740 and 742 during the filling step. The viscosity of the hydrogel precursor also allows the hydrogel precursor to penetrate into the moisture-wicking layer 710 prior to curing, which allows for the resulting cured hydrogel to penetrate or extend into the moisture-wicking layer 710. The ability to pour the hydrogel precursor into the through holes and later cure the precursor allows the hydrogel to fully integrate within the moisture-wicking double-sided adhesive structure 700. As discussed above, at a penetration depth of about 1 to 1.5 mm, there is sufficient integration between the final cured hydrogel and the moisture-wicking layer 710 to form integrated hydrogel portions 750 and 752 and anchor these portions within the moisture-wicking double-sided adhesive structure 700.

The hydrogel precursor can be cured according to various methods depending on the specific chemistry of the hydrogel precursor. Methods for curing the hydrogel precursor can include electron beam photopolymerization and ultraviolet (UV) photopolymerization. Other polymerization methods can be used besides photopolymerization, including thermal polymerization, gamma-ray polymerization, and the like. The curing enables the resulting hydrogel to adhere to the moisture-wicking layer 710, creating strong mechanical and/or chemical bonding between the resulting hydrogel and the interface (e.g., fabric interface) of the moisture-wicking layer 710.

In the case of UV photopolymerization curing, the UV light used generally has a wavelength of 280 to 325 nm for optimum curing. However, the exact wavelength of light used can vary depending on hydrogel monomers and/or polymers used and the photoinitiator chemistry in the hydrogel precursor. UV photopolymerization curing causes the crosslinking process within the hydrogel precursor to take a relatively short period of time and requires no before or after treatment, which eliminates complex processing and waste.

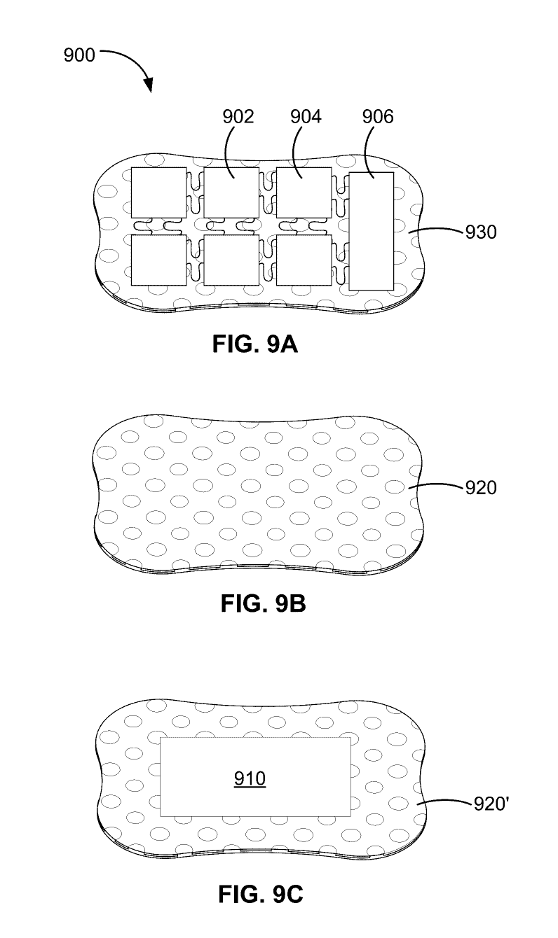

FIGS. 9A-9C are illustrations of perspective views of a moisture wicking double-sided adhesive structure 900 with one or more electronic components 902, 904, 906 affixed thereto and constituting a wearable device in accordance with some embodiments of the invention. The one or more electronic components 902, 904, 906 can be various components as described above, and arranged as a single unit or device islands (as illustrated). According to some aspects, the electronic components 902, 904, 906 can include power sources, communication interfaces, and one or more sensors or sensory platforms related to the sensing that the wearable device performs while affixed to the skin. In some aspects, one or more of the electronic components 902, 904, 906 can be aligned with the electrodes through the moisture wicking double-sided adhesive structure 900, such as one or more hydrogel portions through the moisture wicking double-sided adhesive structure 900. As shown, the one or more electronic components 902, 904, 906 are attached to the moisture wicking double-sided adhesive structure 900 via the adhesion layer 930, which is similar to the second adhesion layer 130 discussed above.

FIG. 9B shows the opposite side of the moisture wicking double-sided adhesive structure 900, specifically the adhesion layer 920, which is similar to the first adhesion layer 120 discussed above.

FIG. 9C shows an alternative arrangement of the opposite side of the moisture wicking double-sided adhesive structure 900 of FIG. 9B. Specifically, the adhesion layer 920′ is formed to have a partial coverage option that exposes the moisture-wicking layer 910 beneath the adhesion layer 920′. Alternatively, the portion illustrated as the moisture-wicking layer 910 can instead be a through hole, and the exposed feature can instead be an electrode, such as a hydrogel portion (e.g., hydrogel portion 750 or 752) described above. Such a hydrogel portion would allow for one or more of the electronic components 902, 904, 906 to be in electrical contact with the skin.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Although any known methods, devices, and materials may be used in the practice or testing of the invention, the methods, devices, and materials in this regard are described herein.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “comprising” or “comprises” is used in reference to compositions, methods, and respective component(s) thereof, that are useful to an embodiment, yet open to the inclusion of unspecified elements, whether useful or not.

As used herein, the term “consisting essentially of” refers to those elements for a given embodiment. The term permits the presence of elements that do not materially affect the basic and novel or functional characteristic(s) of that embodiment of the invention.

As used herein, the term “moisture-wicking” refers to the capability of a material or structure to pull moisture away from a location or a body.

As used herein, the term “breathable” refers to the capability of a material or structure to permit the passage of moisture, vapor or air through the material or structure.

As used herein, the terms “porous” and “porosity” are generally used to describe a structure having a connected network of pores or void spaces (which can, for example, be openings, interstitial spaces or other channels) throughout its volume. The term “porosity” is a measure of void spaces in a material, and is a fraction of volume of voids over the total volume, as a percentage between 0 and 100% (or between 0 and 1).

The terms “flexible” and “bendable” are used synonymously in the present description and refer to the ability of a material, structure, device or device component to be deformed into a curved or bent shape without undergoing a transformation that introduces significant strain, such as strain characterizing the failure point of a material, structure, device or device component. In an exemplary embodiment, a flexible material, structure, device or device component can be deformed into a curved shape without introducing strain larger than or equal to 5%, for some applications larger than or equal to 1%, and for yet other applications larger than or equal to 0.5% in strain-sensitive regions. As used herein, some, but not necessarily all, flexible structures can be also stretchable. A variety of properties provide flexible structures (e.g., device components) of the invention, including material properties such as a low modulus, bending stiffness and flexural rigidity; physical dimensions such as small average thickness (e.g., less than 100 microns, optionally less than 10 microns and optionally less than 1 micron) and device geometries such as thin film and mesh geometries.

As used herein, “stretchable” refers to the ability of a material, structure, device, or device component to be strained (e.g., elongated) without undergoing fracture. In an exemplary embodiment, a stretchable material, structure, device or device component may undergo strain larger than 0.5% without fracturing, for some applications strain larger than 1% without fracturing and for yet other applications strain larger than 3% without fracturing. A used herein, many stretchable structures are also flexible. Some stretchable structures (e.g., device components) are engineered to be able to undergo compression, elongation and/or twisting so as to be able to deform without fracturing. Stretchable structures include thin film structures comprising stretchable materials, such as elastomers; bent structures capable of elongation, compression and/or twisting motion; and structures having an island-bridge geometry. Stretchable device components include structures having stretchable interconnects, such as stretchable electrical interconnects.

As used herein, the term “conformable” refers to a device, material or substrate which has a bending stiffness sufficiently low to allow the device, material or substrate to adopt a desired contour profile, for example a contour profile allowing for conformal contact with a surface having a pattern of relief or recessed features. In certain embodiments, a desired contour profile is that of a tissue in a biological environment, for example skin.

As used herein, the term “conformal contact” refers to contact established between a device and a receiving surface, which can for example be a target tissue in a biological environment. In one aspect, conformal contact involves a macroscopic adaptation of one or more surfaces (e.g., contact surfaces) of a device to the overall shape of a tissue surface. In another aspect, conformal contact involves a microscopic adaptation of one or more surfaces (e.g., contact surfaces) of a device to a tissue surface resulting in an intimate contact substantially free of voids. In some embodiments, conformal contact involves adaptation of a contact surface(s) of the device to a receiving surface(s) of a tissue such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. In some embodiments, the tissue is skin tissue.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A multilayered adhesive structure comprising:
a moisture-wicking layer having a first surface and a second surface;
a first adhesion layer having a first adhesion surface in contact with the first surface of the moisture-wicking layer and a second adhesion surface adapted to contact the skin, wherein the first adhesion layer includes a plurality of pores enabling moisture from the skin to flow to the moisture-wicking layer; and
a second adhesion layer having a third adhesion surface in contact with the second surface of the moisture-wicking layer.

2. The multilayered adhesive structure of claim 1, wherein the second adhesion layer has a fourth adhesion surface in contact with a skin-mounted device.

3. The multilayered adhesive structure of claim 2, wherein the skin-mounted device is selected from the group consisting of an electronic device, a photonic device, an optoelectronic device, or combinations thereof.

4. The multilayered adhesive structure of claim 1, wherein the first surface and the second surface of the moisture-wicking layer are substantially parallel.

5. The multilayered adhesive structure of claim 1, wherein the first adhesion layer comprises a skin adhesive selected from the group consisting of a silicone gel adhesive, a silicone pressure sensitive adhesive, an acrylic pressure sensitive adhesive, a hydrocolloid adhesive, a natural or synthetic rubber adhesive, and a polyurethane based adhesive.

6. The multilayered adhesive structure of claim 1, wherein the moisture-wicking layer comprises a plurality of microchannels and/or nanochannels adapted to promote moisture absorption and transfer via capillary action.

7. The multilayered adhesive structure of claim 1, wherein the moisture-wicking layer comprises a moisture-wicking material selected from the group consisting of absorbent paper, open-cell foam, microfiber, and nanofiber.

8. The multilayered adhesive structure of claim 1, wherein the first adhesion layer and the second adhesion layer each has a width, wherein the width of the first adhesion layer is equal or greater than the width of the second adhesion layer.

9. The multilayered adhesive structure of claim 8, wherein the moisture-wicking layer has a width equal or greater than the width of the second adhesion layer.

10. The multilayered adhesive structure of claim 1, wherein the first adhesion layer includes at least one cutout.

11. The multilayered adhesive structure of claim 1, wherein the first adhesion layer comprises a plurality of disconnected segments.

12. The multilayered adhesive structure of claim 1, further comprising at least one through hole, thereby permitting the skin-mounted device to be electrically connected to the skin.

13. The multilayered adhesive structure of claim 1, wherein the moisture-wicking layer is 50-1,500 μm thick.

14. The multilayered adhesive structure of claim 1, wherein the first adhesion layer is 15-500 μm thick.

15. The multilayered adhesive structure of claim 1, wherein the second adhesion layer is 15-500 μm thick.

16. The multilayered adhesive structure of claim 1, wherein the first adhesion layer is flexible.

17. The multilayered adhesive structure of claim 1, wherein the first adhesion layer is stretchable.

18. The multilayered adhesive structure of claim 1, wherein the first adhesion layer is conformal.

19. A moisture-wicking adhesive structure comprising:
a moisture-wicking layer, comprising a moisture-wicking material, having a first surface and a second surface, and a through hole extending from the first surface and the second surface; and
a hydrogel portion within the through hole,
wherein a perimeter of the hydrogel portion penetrates into the moisture-wicking layer.

20. The adhesive structure of claim 19, wherein the perimeter of the hydrogel portion penetrates about 1 to 1.5 mm into the moisture-wicking layer.

21. The adhesive structure of claim 19, further comprising:
a first adhesion layer having a first adhesion surface in contact with the first surface of the moisture-wicking layer, and a second adhesion surface adapted to affix the adhesive structure to skin.

22. The adhesive structure of claim 21, further comprising:
a second adhesion layer having a third adhesion surface in contact with the second surface of the moisture-wicking layer, and a fourth adhesion surface adapted to affix the adhesive structure to an skin-mounted device.

23. The adhesive structure of claim 22, wherein the fifth adhesion surface of the hydrogel portion and the second adhesion surface of the first adhesion layer are substantially co-planar, and the sixth adhesion surface of the hydrogel portion and the fourth adhesion surface of the second adhesion layer are substantially co-planar.

24. The adhesive structure of claim 19, wherein the hydrogel portion is formed of a hydrogel precursor.

25. The adhesive structure of claim 24, wherein the hydrogel precursor includes one or more monomers, one or more polymers, one or more crosslinking agents, one or more humectants, one or more electrolytes, and water.

26. The adhesive structure of claim 25, wherein the one or more monomers include acrylic acid, a salt of acrylic acid, methacrylic acid, an acrylamide, 2-acrylamido-2-methylpropanesulfonic acid, a salt of 2-acrylamido-2-methylpropanesulfonic acid, dimethyl acrylamide, diacetone acrylamide butyl acrylate, or a combination thereof.

27. The adhesive structure of claim 26, wherein the hydrogel precursor includes 1-25 wt % of the one or more monomers.

28. The adhesive structure of claim 25, wherein the one or more polymers include polyvinylpyrrolidone, poly-2-acrylamido-2-methylpropanesulfonic acid, polyacrylic acid, polyvinyl alcohol, one or more ionic polyacrylamides, one or more non-ionic polyacrylamides, or a combination thereof.

29. The adhesive structure of claim 28, wherein the hydrogel precursor includes 1-50 wt % of the one or more polymers.

30. The adhesive structure of claim 35, wherein the one or more crosslinking agents include N,N′-methylene-bis-acrylamide, 1-hydroxycyclohexyl phenyl ketone, 2-hydroxy-2-methyl-1-phenyl-1-propanone, or a combination thereof.

31. The adhesive structure of claim 30, wherein the hydrogel precursor includes 0.01-5 wt % of the one or more crosslinking agents.

32. The adhesive structure of claim 25, wherein the one or more humectants include glycerol, propylene glycol, triethylene glycol, tripropylene glycol, butylene glycol, or a combination thereof.

33. The adhesive structure of claim 32, wherein the hydrogel precursor includes 1-90 wt % of the one or more humectants.

34. The adhesive structure of claim 25, wherein the one or more electrolytes include sodium chloride, potassium chloride, lithium chloride, or a combination thereof.

35. The adhesive structure of claim 34, wherein the hydrogel precursor includes 0.1-25 wt % of the one or more electrolytes.

36. The adhesive structure of claim 25, wherein the hydrogel precursor includes 1-95 wt % of the water.

37. The adhesive structure of claim 25, wherein the hydrogel precursor includes one or more thickening agents.

38. The adhesive structure of claim 37, wherein the one or more thickening agents include locust bean gum, cellulose, gelatin, agar, alginic acid, casein, collagen, guar gum, or a combination thereof.

39. The adhesive structure of claim 38, wherein the hydrogel precursor includes of 20 wt % or less of the one or more thickening agents.

40. The adhesive structure of claim 19, wherein the moisture-wicking layer includes a plurality of the through hole, and each through hole of the plurality of through holes includes within a hydrogel portion.

41. A method of forming an adhesive structure comprising:
filling one or more through holes formed in a moisture-wicking layer with a hydrogel precursor; and
curing the hydrogel precursor to form one or more hydrogel portions within the moisture-wicking layer,
wherein one or more properties of the hydrogel precursor, one or more characteristics of the filling, or a combination thereof cause the hydrogel precursor to penetrate into the moisture-wicking layer prior to the curing of the hydrogel precursor.

42. The method of forming the adhesive structure of claim 41, wherein the perimeters of the one or more hydrogel portions penetrate about 1 to 1.5 mm into the moisture-wicking layer.

43. The method of forming the adhesive structure of claim 41, wherein the hydrogel precursor has a viscosity of 2000-30000 cPs during the filling of the one or more through holes.

44. The method of forming the adhesive structure of claim 41, wherein the one or more characteristics of the filling include dispensing the hydrogel precursor at a dispenser head speed, a dispenser head height, a flow rate, or a combination thereof to cause the hydrogel precursor to penetrate the moisture-wicking layer.

45. The method of forming the adhesive structure of claim 41, wherein the curing of the hydrogel precursor comprises photopolymerization of the hydrogel precursor.

46. The method of forming the adhesive structure of claim 45, wherein the photopolymerization is electron beam photopolymerization, ultraviolet photopolymerization, or a combination thereof.
